(19) United States
(12) Patent Application Publication
(10) Pub. No.: US 2016/0106100 A1
(43) Pub. Date: Apr. 21, 2016

(54) MICROBICIDAL COMPOSITION COMPRISING AN ISOTHIAZOLONE AND AN AMINE OXIDE

(71) Applicants: Scott BETTS, Manchester (GB); Graham LAWSON, Wirral, Merseyside (GB); Marta URIZAL COMAS, Sant Quirze Del Valles Barcelona (ES); Roman GRABBE, Neustad a.d. Weinstrasse (DE); Rüdiger BAUM, Waghäusel (DE)

(72) Inventors: Scott BETTS, Manchester (GB); Graham Lawson, Merseyside (GB); Marta URIZAL COMAS, Sant Quirze Del Valles Barcelona (ES); Roman GRABBE, Neustadt a.d. Weinstrasse (DE); Rüdiger BAUM, Waghäusel (DE)

(21) Appl. No.: 14/894,416

(22) PCT Filed: May 23, 2014
(86) PCT No.: PCT/EP2014/001394

§ 371 Date: Nov. 27, 2015

(30) Foreign Application Priority Data
May 28, 2013 (EP) 13002757.6

(51) Int. Cl. A01N 43/80; A61K 8/41; A61K 8/49; A61Q 19/00; A01N 33/24; A01N 33/12

(52) U.S. Cl. CPC A01N 43/80; A01N 33/24; A01N 33/12; A61K 8/49; A61Q 19/00; A61K 8/416; A61K 2800/74; A61K 2800/596; A61K 2800/10

(57) ABSTRACT

The present invention relates to a microbicidal composition in the form of a concentrate and to the use of the composition for the preservation of technical and domestic products, preferred for the preservation of wet wipes.

The present invention relates to a microbicidal composition in the form of a concentrate and to the use of the composition for the preservation of technical and domestic products, preferred for the preservation of wet wipes.

The state of the art discloses a large number of active biocides for the preservation of technical and domestical products. For example isothioazolin-3-ones are used as biocides.

EP 1 005 271 B1 discloses a mixture of 2-Methyl-isothiazolinone (MIT) and 1,2-Benzisothiazolinone (BIT). This mixture is sold by Thor GmbH (Speyer, Germany) under the brand name ACTICIDE® MBS.

A combination of an organic amine such as Laurylpropylenediamine and 1,2-Benzisothiazolinone is disclosed in DE 403 32 72 C1. However, according to the teaching of the state of the art, combinations of certain isothiazolinones with organic amines are not storage-stable.

In view of the state of the art, it is an object of the present invention to provide a microbicidal composition in form of a concentrate which is storage stable and methods for inhibiting the growth of microorganisms. A further object of the present invention is to provide a microbicidal composition comprising at least two biocides which inhibit the growth of microorganisms synergistically in the presence of each other.

This object is solved by a microbicidal composition which comprises:
a) as a first component at least one compound selected from the group consisting of 2-methylisothiazolin-3-one (MIT), 1,2-Benzisothiazolin-3-one (BIT) and N-methyl-1,2 Benzisothiazolin-3-one (Me-BIT), and
b) as a second compound at least one amine oxide having the general formula ONR¹R²R³, wherein R¹, R² and R³ are independently selected from C₁ to C₁₈ straight-chained or branched-chained alkyl and C₁ to C₄ hydroxyalkyl, [CH₂CH₂O]ₙH or [CH₂CH₂CH₂O]ₙH, wherein n is an integer from 1 to 4.

The composition of the invention is characterized in that a weight ratio of a) to b) is from 1:20 to 10:1, more preferred from 1:15 to 5:1.

The present invention is based inter alia on the fact that it has surprisingly been found that the component(s) of group a) and the component(s) of group b) interact synergistically, thereby making it possible to lower the required use concentration of the individual compounds. Accordingly, it is possible in numerous fields of application to lower the sensitizing effect of the compositions comprising isothiazolones and to improve their environmental compatibility. At the same time it is possible to lower the costs as compared with known single biocide preparations. A further advantage of a biocide composition of the invention, in addition to its surprisingly broad activity spectrum, lies in its long-term stability and long-term activity.

The microbicidal composition of the invention is particularly suitable for the preservation of water based products. Water based products such as water based building products, cosmetics, polymer emulsions often suffer from microbial infection. Water based products with in an acidic pH range within pH 3 to pH 6.5, preferably within pH 4 to pH 6 will most likely suffer from wet state fungal contamination. In certain products like cosmetic preparations and wet wipes, good technical preservation against fungi is difficult as many fungicidal active ingredients or compositions are too toxic and or too expensive. The microbicidal composition of the invention overcomes these drawbacks as the biocidal compositions shows good technical performance against fungi which should offer a reduced toxicity by the fact that more toxic fungicides are not needed to preserve the preferred the cosmetic/wet wipe application.

The first component a) of the composition of the invention is at least one isothiazolone compound selected from the group consisting of 2-methylisothiazolin-3-one (MIT), 1,2-Benzisothiazolin-3-one (BIT) and N-methyl-1,2 Benzisothiazolin-3-one (Me-BIT). The preferred component (a) is MIT. According to a further preferred embodiment of the invention component a) is MIT, and the presence of further isothiazolones is in this case preferably excluded.

A further development of the invention proposes that the microbicidal composition is essentially free from halogenated isothiazolone. “Essentially” free is taken to mean that the composition has from 0 to at most 3%, preferably from 0 to 1%, and further preferably from 0 to only 0.5%, halogenated isothiazolones, based on the combined weight of halogenated isothiazolone and MIT. Furthermore, it is preferred that the microbicidal composition of the present invention comprises no antimicrobial active amounts of 5-chloro-2-methlyisothiazolin-3-one (CMIT). According to a further preferred embodiment, the microbicidal composition preferably comprises no chlorine-containing isothiazolone.

The concentration of component a) in the concentrate is preferably 0.05% to 15% by weight, more preferably 1 to 10% by weight, in particular 2 to 8% by weight, such as for example 5% by weight (stated as pure active ingredient), based on the weight of the composition.

The at least one organic amine oxide used as component b) is a component having the general formula ONR¹R²R³, wherein R¹, R² and R³ are independently selected from C₁ to C₁₈ straight-chained or branched-chained alkyl and C₁ to C₄ hydroxyalkyl, [CH₂CH₂O]ₙH or [CH₂CH₂CH₂O]ₙH, wherein n is an integer from 1 to 4.

According to a more preferred embodiment of the invention, the at least one amine oxide used as component b) is a component having the general formula ONR¹R²R³ , wherein R¹ and R² are independently selected from C₁ to C₄ alkyl, C₁ to C₄ hydroxyalkyl, [CH₂CH₂O]ₙH or [CH₂CH₂CH₂O]ₙH, wherein n is an integer from 1 to 4, and R³ represents a straight-chained or branched-chained alkyl radical having 8 to 18 C atoms. According to a preferred embodiment of the invention, R¹ and R² are Methyl. According to a further preferred embodiment of the invention, R¹ and R² are Methyl and R³ is a straight chained alkyl radical having 10 to 14 C atoms.

Due to the production process of the at least one organic amine oxide used as component b), the amine oxide contains a certain amount of amine(s) (as a by-product). That means, that the microbicidal composition of the invention comprises from 0 to at most 3%, preferably from 0 to 1%, and further preferably from 0 to only 0.5%, amines, based on the combined weight of amines and amine oxides present in the composition.

According to a preferred embodiment of the invention, the amine oxide, component b) of the invention, is selected from the group consisting of N,N-dimethylcocoalkylamine-N-oxide, N,N-dimethyldecylamine-N-oxide, N,N-dimethyldodecylamine-N-oxide N,N-dicocoalkylmethylamine-N-oxide, N,N-didecylmethylamine-N-oxide, N,N-didodecylmethylamine-N-oxide, N,N-Di-tetradecane-1-methylamine-N-oxide and N,N-Dimethyl-1-tetradecanamine-N-oxide. According to a more preferred embodiment of the invention the amine oxide is N,N-dimethyldodecylamine-N-oxide.

The concentration of component b) in the concentrate is preferably 0.005 to 15% by weight, more preferably 1 to 10% by weight, in particular 2 to 5% by weight, such as for example about 3% by weight, based on the weight of the composition.

According to a further embodiment of the invention, the microbicidal composition also comprises as component c) one or more stabilising agents. Suitable stabilising agents are selected from the group consisting of inorganic oxidizing agents, organic oxidizing agents and transition metals such as salts of Cu. Preferred stabilizing agents, compound(s) c), are inorganic or organic oxidizing agents, such as chloric acid, perchloric acid, bromic acid, iodic acid, periodic acid or salts thereof, nitrates (alkali metal and alkaline earth metal nitrates, such as NaNO₃, KNO₃, Mg(NO₃)₂), H₂O₂, H₂O₂ donors, such as sodium percarbonate, sodium perborate, urea peroxide, peroxides, such as t-butyl peroxide, percarboxyhc acids, such as peracetic acid etc. or salts thereof, persulphuric acids and salts thereof, peroxodisulphonic acid or salts thereof, perphosphoric acids or salts thereof, and transition metals such as salts of Cu, Zn and Fe in relatively high oxidation states. Further examples of compounds which act as stabilizing agents within the context of the invention are also disulphides, such as pyrithion disulphide, oxidases, such as glucose peroxidase, or mixtures of these oxidizing agents.

Preferred stabilizers are potassium iodate, sodium periodate, sodium bromate, H₂O₂, Cu(II) salts (in particular Cu(II) complexes soluble in a weakly alkaline medium at a pH of from 7 to 10). Alternatively, preference is given to oxidizing agents with an independent biocidal effectiveness, such as oxidases, H₂O₂, peracetic acid, and pyrithone disulphide.

Particularly preferred stabilizers are potassium iodate, sodium bromate, H₂O₂ and Cu(II) complexes, where potassium iodate or H₂O₂ is very particularly preferred as stabilizing agent.

The concentration of the stabilizing agent c) (if present) in the concentrate is preferably 0.01 to 10% by weight, more preferably 0.05 to 1% by weight, in particular 0.08 to 0.5% by weight, such as for example 0.1 to 0.3% by weight, based on the weight of the composition.

According to a further preferred embodiment of the invention, the microbicidal composition comprises as a further component d) a quaternary ammonium compound. Quaternary ammonium compounds, also known as quats, comprise a positively charged ion of the structure NR₄⁺, R being an alkyl or an aryl group. According to a more preferred embodiment, the quaternary ammonium compound is having the general structure R¹R²R³R⁴N⁺A⁻, wherein:

R¹ and R² are independently selected from a straight-chained or branched-chained alkyl residue having from 1 to 22 carbon atoms or a phenalkyl residue having from 1 to 3 carbon atoms in the alkyl residue,

R³ and R⁴ are independently selected from a straight-chained or branched-chained alkyl residue having from 1 to 22 carbon atoms,

and A⁻ is a counterion, preferably a counterion selected from the group consisting of chloride, iodide, bromide, hydrogensulfate, lactate, citrate, carbonate and bicarbonate.

Preferably, R², R³ and R⁴ or R³ and R⁴ each represent methyl, ethyl, 2-hydroxyethyl or 2-hydroxypropyl, more preferred R², R³ and R⁴ or R³ and R⁴ represent a methyl group.

According to a preferred embodiment of the invention, the quaternary ammonium compound is selected from the group consisting of Behentrimonium chloride (CH₃(CH₂)₂₁N(Cl )(CH₃)₃), Cetyltrimethylammoniumchloride (CH₃(CH₂)₁₅N(Cl)(CH₃)₃), Cetyltrimethylammoniumbromide (CH₃(CH₂)₁₅N(Br)(CH₃)₃, Di-n-decyl-dimethylammonium chloride (CH₃(CH₂)₉)₂N(Cl)(CH₃)₂, Dioctyl-dimethylammonium chloride (CH₃(CH₂)₇)₂N(Cl)(CH₃)₂, N-Decyl-N-isononyl-N,N-dimethylammonium chloride (CH₃(CH₂)₉)(C₉H₁₉)N(Cl )(CH₃)₂, Dimethyl-enzylammonium chloride, N,N-Didecyl-N,N-dimethyl ammonium propionate, Cotrimethyl ammonium chloride, Benzyl-cocoalkyl-dimethylammonium chlorides such as (C₆H₅)CH₂N(Cl )(CH₃)₂(C₁₂H₂₅) or (C₆H₅)CH₂N(Cl )(CH₃)₂(C₁₄H₂₉) or (C₆H₅)CH₂N(Cl )(CH₃)₂(C₁₆H₃₃), Methyl benzethonium chloride, Benzethonium chloride (N-Benzyl-N,N-dimethyl-2{-2-{4-(2,4,4-trimethylpentan 2-yl)phenoxy]ethoxy}ethanaminium chloride), Cetylpyridinium chloride and Domiphenbromide.

According to a further preferred embodiment of the invention the quaternary ammonium compound is a benzalkonium compound having the general structure C₆H₅CH₂N(CH₃)₂—R^{1 A}⁻, wherein R is (CₙH₂n+1), n is 8 to 18 and A⁻ is a counterion. According to a more preferred embodiment of the invention, the benzalkonium compound is selected from the group consisting of benzalkonium chloride, benzalkonium bromide, benzalkoium carbonate, benzalkonium saccharinate , benzalkonium hydroxide and benzalkonium propionate. As component d), preference is given to benzalkonium chloride.

According to a preferred embodiment, the quaternary ammonium compound is selected from the group consisting of benzalkonium chloride, benzalkonium bromide, benzalkoium carbonate, Behentrimonium chloride, Cetyltrimethylammoniumchloride, Cetyltrimethylammoniumbromide, Di-n-decyl-dimethylammonium chloride and Benzethonium chloride.

The concentration of component d) (if present) in the concentrate is preferably 0.001 to 15% by weight, more preferably 1 to 10% by weight, in particular 2 to 5% by weight, such as for example about 3% by weight, based on the weight of the composition.

The composition according to the invention optionally comprises e) solvents and/or f) further microbicidal active ingredients, functional additives or auxiliaries.

Suitable solvents, component e) are water, alcohols, such as ethanol, propanol, benzyl alcohol, glycols such as ehtlyene glycol, propylene glycols such as dipropylene glycol, butanediols, glycol ethers, such butyl glycol, butyl diglycol, phenoxyethanol, phenoxypropanols, such as glycerol, alcanediols and alkyl glycerol ethers and mixtures thereof. Preference is given to using VOC (volatile organic compounds)—free or low VOC solvents. The solvent is preferably water.

The concentration of the solvent, component e), in the concentrate is preferably 40 to 98.99% by weight, more preferably 60 to 98% by weight, in particular 80 to 96% by weight, such as for example 88 to 90% by weight. The concentrate preferably comprises water in an amount of from 40 to 98.99% by weight, more preferably 60 to 98% by weight, in particular 80 to 96% by weight, such as for example 88 to 90% by weight.

Suitable functional additives, component f), are: complexing agents, such as EDTA, NDA, thickeners, fillers, antioxidants, such as vitamin E, BHA and BHT, alkalizing agents, such as NaOH, KOH, alkali metal carbonate, alkali metal hydrogen carbonate, ammonia, low molecular weight amines or alkanolamines, acidifying agents, such as carboxylic acids, such as acetic acid, preferably hydroxy carboxylic acids, such as lactic acids, citric acid, buffers, corrosion inhibitors (such as benzotriazole), wetting agents and low-temperature stabilizers.

The concentrates according to the invention are preferably clear and homogenous and are in the form of liquids, preferably aqueous solutions. They are adequately colour-stable, low-temperature-stable, storage-stable and active-ingredient-stable. According to a further embodiment of the invention, the concentrates according to the invention are in form of semi-solid, pasty compositions.

The pH of the concentrate is preferably in the range of from 2 to 5, more preferably in the range of from 2.5 to 4.

Preference is given to a liquid concentrate which comprises:
(a) at least one compound selected from the group consisting of MIT, BIT and Me-BIT, and
(b) at least one amine oxide having the general formula ONR¹R²R³, wherein R¹, R² and R³ are independently selected from C₁ to C₁₈ straight-chained or branched-chained alkyl and C₁ to C₄ hydroxyalkyl, [CH₂CH₂O ]ₙH or [CH₂CH₂CH₂O]ₙH, wherein n is an integer from 1 to 4 ,
(c) at least one selected stabilizing agent,
(d) at least one quaternary ammonium compound (as defined above),
(e) at least one solvent (preferably water) and
(f) optionally further microbicidal active ingredients, functional additives or auxiliaries.

The present invention also relates to the use of the microbicidal composition, comprising the components (a), (b) and (c), or comprising the components (a), (b) and (d), or comprising the components (a), (b), (c) and (d) for the preservation of technical or domestic products. Accordingly, the invention relates to a technical or domestic product which preferably comprises 0.001 to 1.0% by weight, more preferably 0.01 to 0.5% by weight of the composition according to the invention.

Examples of products are preferably water based or water-dilutable products of all types. Examples of products are technical products selected from polymer dispersions, paints, adhesives, paper coating compositions, textile softening and sizing compositions, cleaning and polishing compositions, cosmetic and pharmaceutical preparations, household products, pack preservatives, spinning baths, cooling lubricants, leather treatment compositions, wet wipes and silicone and bitumen emulsions.

The synergistically effective components (a) and (b) or (a), (b) and (d) may—preferably—be present in a product, e.g. a liquid concentrate, and be dosed into the end product or intermediate product to be preserved in a known manner. The synergistically effective components (a) and (b) or (a), (b) and (d) may, however, also be present in two or more separate products (solid and/or liquid) and be dosed into the end product or intermediate product to be preserved in a known manner.

The product, e.g. the application solution thus comprises for example:
(a) 25 to 300 ppm of isothiazolone (as defined above), preferably 50 to 150 ppm of isothiazolone and
(b) 30 to 500 ppm amine oxide (as defined above), preferably 50 to 300 ppm of amine oxide, and, if present
(c) 25 to 250 ppm of the quaternary ammonium compound (as defined above), preferably 75 to 250 ppm of the quaternary ammonium compound.

According to a preferred embodiment of the invention, the product, e.g. the application solution thus comprises:
(a) 25 to 300 ppm of 2-methylisothiazolin-3-one, preferably 50 to 150 ppm of 2-methylisothiazolin-3-one and
(b) 30 to 500 ppm of N,N-dimethyldodecylamine-N-oxide, preferably 50 to 300 ppm of N,N-dimethyldodecylamine-N-oxide, and
(c) 25 to 250 ppm of benzalkonium chloride, preferably 75 to 250 ppm of benzalkonium chloride.

Preferred products are low anionic surfactants and preferably comprise less than 5% by weight of anionic surfactant.

Examples of technical or domestic products are polymer dispersions, paints, adhesives, paper coating compositions, textile softening and sizing compositions, cleaning and polishing compositions, cosmetic and pharmaceutical preparations, household products, pack preservatives, spinning baths, cooling lubricants, leather treatment compositions, wet wipes and silicone and bitumen emulsions.

The composition of the invention is characterized in that a weight ratio of a) to b) to d) is from 1:10 to 10:1 to 1:10 to 10:1 to 1:10 to 10:1, more preferred from 1:5 to 5:1 to 1:5 to 5:1 to 1:5 to 5:1.

### EXAMPLES

The following examples and the applications are intended to illustrate the invention more closely but without restricting it thereto. All percentage details are percentages by weight (% by weight), if not explicitly stated otherwise.

Investigation of the Synergistic Effect

The synergism of a combination of 2-Methyl-isothiazolinone (MIT) with N,N-Dimethyldodecan-l-amine oxide was tested. Test organisms used was the mould Aspergillus niger (DSM 12623). For the purpose of the test, mixtures containing different concentrations of MIT and N,N-Dimethyldodecan-1-amine oxide were prepared and tested for their action on Aspergillus niger. The active compound mixtures in the corresponding concentrations were introduced into selective nutrient media (Sabouraud glucose agar for Aspergillus niger and the test organisms were likewise incorporated into the agar. The cell density in the agar was in each case 3.7·10⁶ spores per ml for Aspergillus niger. The incubation time is 72 hours at 25° C. This was followed by visual testing for growth of Aspergillus niger. The growth was apparent through the formation of colonies or mycelia on the surface of the agar. In this way the minimum inhibitory concentrations (MICs) of the two active compounds were determined, individually and in combination. The MIC is the concentration at which there is no formation of colonies or mycelia on the surface of the agar ( Aspergillus niger).

The synergism that occurred was presented numerically by calculation of the synergy index (SI). This calculation was made using the common method of F. C. Kull et al., Applied Microbiology, vol. 9 (1961), p. 538. SI is calculated there in accordance with the following formula:

Synergy index SI=Qa/QA+Qb/QB

In the application of this formula to the MIT+N,N-Dimethyldodecan-1-amine oxide biocide system tested here, the variables in the formula have the following definition:

Qa=concentration of MIT in the mixture of N,N-Dimethyldodecn-1-amine oxide+MIT

QA=concentration of MIT as sole biocide

Qb=concentration of N,N-Dimethyldodeca-1-amine oxide in the biocide mixture of

N,N-Dimethyldodecan-1-amine oxide+MIT

QB=concentration of N,N-Dimethyldodecan-1-amine oxide as sole biocide

If the synergy index has a value of more than 1, this means that there is an antagonism. If the synergy index adopts a value of 1, this means that the action of the two biocides is additive. If the synergy index takes on a value of below 1, this means that there is synergism of the two biocides.

### Example 1

Calculation of the synergy index of N,N-Dimethyldodecan-1-arnine oxide+MIT in respect of Aspergillus niger for an incubation time of 72 hours at 25° C.

TABLE 1

| Biocide | | | | | | | | | | | | | | | | | | | | | | | | | | |
|---|---|---|---|---|---|---|---|---|---|---|---|---|---|---|---|---|---|---|---|---|---|---|---|---|---|---|
| Concen- | | | | | | | | | | | | | | | | | | | | | | | | | | |
| trations | | | | | | | | | | | | | | | | | | | | | | | | | | |
| Euroxide | Growth (+)/No Growth (−) in Tubes - ACTICIDE ® M 20 | | | | | | | | | | | | | | | | | | | | | | | | | |
| (%) | 0 | | 0.005 | | 0.01 | | 0.02 | | 0.03 | | 0.04 | | 0.06 | | 0.08 | | 0.1 | | 0.2 | | 0.3 | | 0.4 | | 0.5 | |
| 0 | + | + | + | + | + | + | + | + | + | + | + | + | + | + | + | + | + | + | + | + | + | + | + | + | − | − |
| 0.005 | + | + | + | + | + | + | + | + | + | + | + | + | − | − | − | − | − | − | − | − | − | − | − | − | − | − |
| 0.01 | + | + | + | + | + | + | + | + | + | + | − | − | − | − | − | − | − | − | − | − | − | − | − | − | − | − |
| 0.015 | + | + | + | + | + | + | + | + | − | − | − | − | − | − | − | − | − | − | − | − | − | − | − | − | − | − |
| 0.02 | + | + | + | + | + | + | − | − | − | − | − | − | − | − | − | − | − | − | − | − | − | − | − | − | − | − |
| 0.025 | + | + | + | + | + | + | − | − | − | − | − | − | − | − | − | − | − | − | − | − | − | − | − | − | − | − |
| 0.03 | + | + | + | + | − | − | − | − | − | − | − | − | − | − | − | − | − | − | − | − | − | − | − | − | − | − |
| 0.05 | + | + | − | − | − | − | − | − | − | − | − | − | − | − | − | − | − | − | − | − | − | − | − | − | − | − |
| 0.07 | − | − | − | − | − | − | − | − | − | − | − | − | − | − | − | − | − | − | − | − | − | − | − | − | − | − |

Growth Ratings:
+ = Growth
− = No growth

Euroxide® LO/A: a composition consisting of 31% C-12 Amine oxide (CAS No. 1643-20-5), +0.7%(max) Dimethyl lauramine (Cas No. 84649-84-3) in water.

ACTICIDE® M 20: a composition consisting of 20% by weight of MIT in water.

TABLE 2
Synergy Index Table - Aspergillus niger

| | N,N-Dimethyldodecan- | Synergy Index |
|---|---|---|
| MIT conc. (ppm) | 1-amine oxide (ppm) | (Qa/QA + Qb/QB) |
| 0 | 231 (MIC) | 1.00 |
| 10 | 165 | 0.72 |
| 20 | 99 | 0.45 |
| 40 | 66 | 0.33 |
| 60 | 49.5 | 0.27 |
| 80 | 33 | 0.22 |
| 120 | 16.5 | 0.19 |
| 1000 (MIC) | 0 | 1.00 |

From Table 2 it is evident that the optimum synergy, i.e. the lowest synergy index (0.22) of a biocide composition composed of MIT and N,N-Dimethyldodecan-1-amine oxide, is present at 33 ppm by weight N,N-Dimethyldodecan-1-amine oxide and 80 ppm MIT.

Furthermore, the synergism of a combination of 2-Methyl-isothiazolinone (MIT) with N,N-Dimethyldodecan-1-amine oxide was tested using Candida albicans (ATCC 10231). For the purpose of the test, mixtures containing different concentrations of MIT and N,N-Dimethyldodecan-1-amine oxide were prepared and tested for their action on Candida albicans. The active compound mixtures in the corresponding concentrations were introduced into selective nutrient media (Muller Hinton Broth). The cell density in the broth was in each case 1.0-10⁶ spores per ml. The incubation time is 72 hours at 25° C. This was followed by visual testing for growth of Candida albicans. In this way the minimum inhibitory concentrations (MICs) of the two active compounds were determined, individually and in combination. The synergism that occurred was presented numerically by calculation of the synergy index (SI). Results are shown in Table 3.

TABLE 3

| MHK (MIT) | MHK (Amineoxide) | | Synergistic |
|---|---|---|---|
| [ppm] | [ppm] | Total [ppm] | Index |
| 125 | 0 | 125 | 1.00 |
| 75 | 5 | 80 | 0.70 |
| 75 | 10 | 85 | 0.80 |
| 50 | 15 | 65 | 0.70 |
| 40 | 20 | 60 | 0.72 |
| 30 | 25 | 55 | 0.74 |
| 30 | 30 | 60 | 0.84 |
| 20 | 35 | 55 | 0.86 |
| 10 | 40 | 50 | 0.88 |
| 0 | 50 | 50 | 1.00 |

Amineoxide: N,N-Dimethyldodecan-1-amine oxide

From Table 3 it is evident that the optimum synergy, i.e. the lowest synergy index (0.70) of a biocide composition composed of MIT and N,N-Dimethyldodecan-1-amine oxide, is present at 15 ppm by weight N,N-Dimethyldodecan-1-amine oxide and 15 ppm MIT.

In a further Experiment the synergy index of a ternary mixture of Benzalkoniumchloride, N,N-Dimethyldodecan-1-amine oxide and MIT was determined in respect of Aspergillus niger for an incubation time of 72 hours at 25 ° C.

The synergy of an active substance mixture that contains Benzalkoniumchloride in addition to N,N-Dimethyldodecan-1-amine oxide and MIT, against the microorganism Aspergillus niger is shown as in Table 4. The table below shows the MIC values of the tested biocide compositions. The MIC value was 82.75 ppm when N,N-Dimethyldodecan-1-amine oxide and MIT alone were used, and 80 ppm when Benzalkoniumchloride alone was used.

TABLE 4

| MHK (MIT + Amine oxide) | MHK (BAC) | |
|---|---|---|
| (weight ratio 8:1) [ppm] | [ppm] | Synergistic Index |
| 82.75 | 0 | 1.00 |
| 41.38 | 30 | 0.88 |
| 41.38 | 35 | 0.94 |
| 15.72 | 40 | 0.69 |
| 13.24 | 45 | 0.72 |
| 13.24 | 50 | 0.79 |
| 0 | 80 | 1.00 |

Amine oxide: N,N-Dimethyldodecan-1-amine oxide
BAC: Benzalkoniumchloride

From Table 4 it is evident that the optimum synergy, i.e. the lowest synergy index (0.69) of a biocide composition composed of MIT, Benzalkoniumchloride and N,N-Dimethyldodecan-1-amine oxide, is present at 15.72 ppm by weight N,N-Dimethyldodecan-1-amine oxide/MIT and 40 ppm Benzalkoniumchloride.

### Example 2

Preservation of a Skin Cream Formulations (Cosmetic Formulation)

To examine the preservation efficacy of the biocidal composition of the invention eight skin cream formulations were tested and labelled as follows:
Skin Cream 0.10% Microcare® MT
Skin Cream 0.50% MIT/AO Blend
Skin Cream 0.75% MIT/AO Blend
Skin Cream 1.00% MIT/AO Blend
Skin Cream 0.50% MIT/AO/BAC Blend
Skin Cream 0.75% MIT/AO/BAC Blend
Skin Cream 1.00% MIT/AO/BAC Blend
Skin Cream Unpreserved (control)
Skin cream: water bases skin cream composition (comprising water, glycerol, steareth-21, mineral oil, cetyl alcohol, cetyl dimethicone, Steareth-2 and Perfume
Microcare® MT: a composition comprising 9,6% by weight of MIT in water.
MIT/AO Blend: a composition comprising 5% ACTICIDE® M20, 10% Euroxide® LO/A, 10% monopropylene glycol and 75% water
MIT/AO/BAC Blend a composition comprising 5% ACTICIDE® M20, 10% Euroxide® LO/A, 10% monopropylene glycol, 5% ACTICIDE® BAC5OM and 70% water.

20g aliquots of the formulation were inoculated individually using a single inoculum of bacteria or fungi. Inoculated samples were incubated at 25° C. and 1 gram removed after 0 hours, 7, 14 and 28 days. A plate count was performed on these samples with Letheen broth being used as the inactivator. Where applicable, serial dilutions were carried out using ¼ strength Ringer solution. Samples inoculated with bacteria were plated onto Tryptone Soya Agar and incubated at 30° C.±2° C. for 72 hours. Those samples inoculated with fungi were plated onto Sabouraud Dextrose Agar and incubated at 25° C.±2° C. for 5 days. The Total Viable Count per gram was then calculated using the dilution factor and recorded. The Inocula used were:

| | |
|---|---|
| Aspergillus niger | IMI 149007 |
| Candida albicans | NCPF 3179 |
| Pseudomonas aeruginosa | ATCC 9027 |
| Staphylococcus aureus | ATCC 6538 |

IMI = International Mycological Institute
NCPF = National Collection of Pathogenic Fungi
ATCC = American Type Culture Collection

Results are detailed in the Tables below:

Challenge Test Results Test Method Total Viable Counts

| Organism | Description | 0 hour | 2 days | 7 days | 14 days | 28 days |
|---|---|---|---|---|---|---|
| A. niger | Skin Cream 0.10% Micro- | 2.1 × 10⁵ | | | 4.1 × 10³ | 2.2 × 10² |
| | care ® MT | | | | | |
| | Skin Cream 0.50% | 2.6 × 10⁵ | | | 2.7 × 10³ | 2.1 × 10³ |
| | MIT/AO Blend | | | | | |
| | Skin Cream 0.75% | 2.6 × 10⁵ | | | 7.0 × 10² | 3.3 × 10² |
| | MIT/AO Blend | | | | | |
| | Skin Cream 1.00% | 2.5 × 10⁵ | | | 5.2 × 10³ | 1.9 × 10³ |
| | MIT/AO Blend | | | | | |
| | Skin Cream 0.50% | 1.8 × 10⁵ | | | 2.3 × 10² | <20 |
| | MIT/AO/BAC Blend | | | | | |
| | Skin Cream 0.75% | 3.1 × 10⁵ | | | 1.9 × 10² | <20 |
| | MIT/AO/BAC Blend | | | | | |
| | Skin Cream 1.00% | 2.6 × 10⁵ | | | 1.6 × 10² | <20 |
| | MIT/AO/BAC Blend | | | | | |
| | Skin Cream Unpreserved | 2.6 × 10⁵ | | | 1.6 × 10⁵ | 1.2 × 10⁵ |
| C. albicans | Skin Cream 0.10% Micro- | 5.5 × 10⁵ | | | 4.3 × 10⁴ | 3.9 × 10⁴ |
| | care ® MT | | | | | |
| | Skin Cream 0.50% | 6.9 × 10⁵ | | | 4.7 × 10⁴ | 3.67 × 10⁵ |
| | MIT/AO Blend | | | | | |
| | Skin Cream 0.75% | 5.5 × 10⁵ | | | 5.2 × 10³ | 4.4 × 10³ |
| | MIT/AO Blend | | | | | |
| | Skin Cream 1.00% | 6.8 × 10⁵ | | | 6.6 × 10² | 5.0 × 10¹ |
| | MIT/AO Blend | | | | | |
| | Skin Cream 0.50% | 4.3 × 10⁵ | | | <20 | <20 |
| | MIT/AO/BAC Blend | | | | | |
| | Skin Cream 0.75% | 4.7 × 10⁵ | | | <20 | <20 |
| | MIT/AO/BAC Blend | | | | | |
| | Skin Cream 1.00% | 6.7 × 10⁵ | | | <20 | <20 |
| | MIT/AO/BAC Blend | | | | | |
| | Skin Cream Unpreserved | 5.7 × 10⁵ | | | 3.0 × 10⁶ | 2.3 × 10⁶ |

| Organism | Description | 0 hour | 2 days | 7 days | 14 days | 28 days |
|---|---|---|---|---|---|---|
| Ps. aeruginosa | Skin Cream 0.10% Micro- | 2.3 × 10⁶ | 3.8 × 10³ | <20 | | <20 |
| | care ® MT | | | | | |
| | Skin Cream 0.50% | 2.6 × 10⁶ | 1.5 × 10⁴ | <20 | | <20 |
| | MIT/AO Blend | | | | | |
| | Skin Cream 0.75% | 1.6 × 10⁶ | 3.1 × 10³ | <20 | | <20 |
| | MIT/AO Blend | | | | | |
| | Skin Cream 1.00% | 2.1 × 10⁶ | 1.6 × 10⁴ | <20 | | <20 |
| | MIT/AO Blend | | | | | |
| | Skin Cream 0.50% | 3.2 × 10⁵ | 1.0 × 10¹ | <20 | | <20 |
| | MIT/AO/BAC Blend | | | | | |
| | Skin Cream 0.75% | 3.5 × 10⁴ | <20 | <20 | | <20 |
| | MIT/AO/BAC Blend | | | | | |
| | Skin Cream 1.00% | 9.4 × 10³ | <20 | <20 | | <20 |
| | MIT/AO/BAC Blend* | | | | | |
| | Skin Cream Unpreserved | 1.8 × 10⁶ | 4.1 × 10⁵ | 1.2 × 10⁶ | | 4.7 × 10⁶ |
| S. aureus | Skin Cream 0.10% Micro- | 5.3 × 10⁶ | 1.6 × 10⁵ | <20 | | <20 |
| | care ® MT | | | | | |
| | Skin Cream 0.50% | 4.9 × 10⁶ | 4.3 × 10⁶ | 2.0 × 10² | | <20 |
| | MIT/AO Blend | | | | | |
| | Skin Cream 0.75% | 5.3 × 10⁶ | 2.1 × 10⁶ | 5.0 × 10¹ | | <20 |
| | MIT/AO Blend | | | | | |
| | Skin Cream 1.00% | 2.8 × 10⁶ | 2.7 × 10⁶ | 4.0 × 10¹ | | <20 |
| | MIT/AO Blend | | | | | |
| | Skin Cream 0.50% | 6.3 × 10⁶ | <20 | <20 | | <20 |
| | MIT/AO/BAC Blend | | | | | |
| | Skin Cream 0.75% | 3.7 × 10⁶ | <20 | <20 | | <20 |
| | MIT/AO/BAC Blend | | | | | |
| | Skin Cream 1.00% | 8.2 × 10⁶ | <20 | <20 | | <20 |
| | MIT/AO/BAC Blend | | | | | |
| | Skin Cream Unpreserved | 8.1 × 10⁶ | 1.9 × 10⁶ | 1.1 × 10⁵ | | 1.0 × 10⁵ |

*Inactivator check was carried out on this sample and found to be valid in Letheen Broth.

| | | Inoculum in |
|---|---|---|
| Organism | Inoculum | Sample |
| Aspergillus niger IMI 149007 - F35 | 4.0 × 10⁷ | 4.0 × 10⁵ |
| Candida albicans NCPF 3179 - F82 | 7.5 × 10⁷ | 7.5 × 10⁵ |
| Pseudomonas aeruginosa ATCC 9027 - B193 | 1.0 × 10⁸ | 1.0 × 10⁶ |
| Staphylococcus aureus ATCC 6538 - B230 | 2.0 × 10⁸ | 2.0 × 10⁶ |

### Example 3

### Stability of a Product of the Invention

To check the stability and physical properties of the product of the invention, preferably a product for the preservation of wet wipes, based on MIT +Benzalkoniumchloride and +N,N-Dimethyldodecan-1-amine oxide, the following formulation was prepared:

| | % |
|---|---|
| Microcare ® MT | 10.3 |
| Oxidet DM-20 | 10.0 |
| Monopropyleneglycol | 10.0 |
| ACTICIDE ® BAC 50 M | 5.0 |
| Potassium Iodate | 0.01 |
| Water | 64.7 |
| | 100.0 |

Oxidet ® DM-20: Lauramine oxide.

The stability of the product has been satisfactory carried out at 20° C. and 40° C. after 1 month.

| t/weeks | | | | |
|---|---|---|---|---|
| 0 | 1 | 2 | 3 | 4 |

T [° C.] 20° C.

| | | | | | |
|---|---|---|---|---|---|
| pH | 7.2 | 7.2 | 7.2 | 7.1 | 7.2 |
| ρ (g/ml) | 0.997 | | | | 0.998 |
| IR (nD) | 1.3554 | | | | 1.3553 |
| A.C. (%) | 2.7 | | | | 2.6 |
| MIT (%) | 1.0 | 1.0 | 1.0 | 1.0 | 1.0 |

T [° C.] 40° C.

| | | | | | |
|---|---|---|---|---|---|
| pH | 7.2 | 7.2 | 7.1 | 7.2 | 7.2 |
| ρ (g/ml) | 0.997 | | | | 0.999 |
| IR (nD) | 1.3554 | | | | 1.3552 |
| A.C. (%) | 2.7 | | | | 2.6 |
| MIT (%) | 1.0 | 1.0 | 1.0 | 1.0 | 1.0 |

### Example 4

### Preservation of Wet Wipes

To test the new preservative combination based on N,N-Dimethyldodecan-1-amine oxide+MIT+Benzalkoniumchloride, the combination was applied in wet wipes at 1.0% in a 100% viscose non-woven.

Solution 1

1% MIT
10% of a 30% N,N-Dimethyldodecan-1-
amine oxide
10% MPG
5% ACTICIDE ® BAC 50
Balance Water

We used a 100% viscose non-woven to impregnate the liquor with solution 1.

The test method comprises the inoculation of a product with blended microbial cultures of known strains (pools) and monitoring the number of survivors at set time points over a period of 28 days incubation at constant temperature. A set of acceptance criteria based on reduction in viability are used to interpret the results. It is recommended to test three groups of microorganisms, bacteria, moulds and yeasts. The inocula were made in a solution of peptone for bacteria and yeasts, and in peptone water (peptone with polysorbate 80) for mould. The method of inoculation by streaking serves to quantify the growth of microorganisms on a medium TSA (Tryptone Soy Agar) for bacteria and Sabouraud medium for yeasts and moulds.

Inoculums

| | Thoma | In the extract |
|---|---|---|
| | cell | solution |
| INOCULUM | CFU/ML | CFU/ml |
| Bacteria Pool | 3.0 × 10⁸ | 2.0 × 10⁵ |
| Pseudomonas aeruginosa ATCC 9027 | | |
| Pseudomonas putida ATCC 31483 | | |
| Burkholderia cepacia ATCC 25416 | | |
| Klebsiella pneumoniae ATCC 10031 | | |
| Enterobacter gergoviae ATCC 33028 | | |
| Escherichia coli ATCC 8739 | | |
| Staphylococcus aureus ATCC 6538 | | |
| Staphylococcus epidermidis ATCC 12228 | | |
| Yeasts Pool | 4.8 × 10⁷ | 2.0 × 10⁴ |
| Candida albicans ATCC 10231 | | |
| Candida parapsilosis ATCC 22019 | | |
| Moulds Pool | 2.1 × 10⁷ | 2.0 × 10⁴ |
| Aspergillus brasiliensis ATCC 16404 | | |
| Penicillium funiculosum ATCC 36839 | | |
| Gliocladium virens ATCC 9645 | | |

Sample Description

| | | | | Inoculum |
|---|---|---|---|---|
| | | Size of 1 | Weight of | volume = |
| | Weight of | wipe [cm²] | 3 wipes | W/2 |
| Sample | 1 wipe [g] | (Surface) | [g] | [mL] |
| Solution 1 - 1% | 8.8 | 360 | 26.4 | 7.9 |
| (100% vis.) | | | | |

Anti-Microbial Preservation Efficacy Results

| | | Theoretical | | | | |
|---|---|---|---|---|---|---|
| ORGANISMS | Sample | value | DAY 0 | DAY 7 | DAY 14 | DAY 28 |
| Bacteria pool | Solution 1 - 1% | 2.0 × 10⁵ | 4.2 × 10⁴ | <200 | <200 | <200 |
| | (100% vis.) | | | | | |
| Yeasts pool | Solution 1 - 1% | 2.0 × 10⁴ | 1.8 × 10⁴ | | <200 | <200 |
| | (100% vis.) | | | | | |
| Moulds pool | Solution 1 - .1% | 2.0 × 10⁴ | 1.2 × 10⁴ | | <200 | <200 |
| | (100% vis.) | | | | | |

Interpretation of the Results
Bacteria: Log reduction >3 by 7 days and continued reduction to non-detectable level by end of test.
Moulds and Yeasts: Log reduction >2 after 14 days and continued reduction to nondetectable level by end of test.

1. Microbicidal composition which comprises:
a) as a first component at least one compound selected from the group consisting of 2-methylisothiazolin-3-one (MIT), 1,2-Benzisothiazolin-3-one (BIT) and N-methyl-1,2 Benzisothiazolin-3-one (Me-BIT), and
b) as a second compound at least one amine oxide having the general formula having the general formula ONR R R , wherein R , R and R are independently selected from Q to C₁₈ straight-chained or branched-chained alkyl and d to C₄ hydroxyalkyl, [CH₂CH₂0]ₙH or [CH₂CH₂CH₂0)]ₙH, wherein n is an integer from 1 to 4, characterized in that a ratio of a) to b) is from 1:20 to 10:1.

2. Composition according to claim 1, characterized in that it also comprises c) one or more stabilizing agents selected from the group consisting of inorganic oxidizing agents, organic oxidizing agents and transition metals such as salts of Cu.

3. Composition according to claim 1, characterized in that the amine oxide is selected from the group consisting of N,N-dimethylcocoalkylamine oxide, N,N-dimethyldecylamine-N-oxide, N,N-dimethyldodecylamine-N-oxide N,N-dicocoalkylmethylamine-N-oxide, N,N-didecylmethylamine-N-oxide, N,N-didodecylmethylamine-N-oxide, N,N-Di-tetradecane-1-methylamine-N-oxide and N,N-dimethyl-1-tetradecanamine-N-oxide.

4. Composition according to claim 1, characterized in that the amount of component a) is 0.005% to 15% by weight.

5. Composition according to claim 1, characterized in that the amount of component b) is 0.005%) to 15% by weight.

6. Composition according to claim 1, characterized in that the amount of component c) is 0.001 to 15% by weight.

7. Composition according to claim 1, characterized in that it also comprises d) one or more quaternary ammonium compounds.

8. Composition according to claim 7, characterized in that the quaternary ammonium compound is having the general structure R¹R²R³RN⁺A″, wherein:
R¹ and R² are independently selected from a straight-chained or branched-chained alkyl residue having from 1 to 22 carbon atoms or a phenalkyl residue having from 1 to 3 carbon atoms in the alkyl residue,
R³ and R⁴ are independently selected from a straight-chained or branched-chained alkyl residue having from 1 to 22 carbon atoms, and A″ is a counterion.

9. Composition according to claim 7, characterized in that the quaternary ammonium compound is a benzalkonium compound having the general structure C₆H₅CH₂N(GH₃)₂—R⁺A″, wherein R is (CₙH₂n+1), n is 8 to 18 and A″ is an counter- ion.

10. Composition according to claim 7, characterized in that the quaternary ammonium compound is selected from the group consisting of is selected from the group consisting of Behentrimonium chloride, Cetyltrimethylammoniumchloride, Cetyltrimethylammoniumbromide, Di-n-decyl-dimethylammonium chloride, Dioctyl-dimethylammonium chloride, N-Decyl-N-isononyl-N,N-dimethylammonium chloride, Dimethyl-enzylammonium chloride, N,N-Didecyl-N,N-dimethyl ammonium propionate, Cotrimethyl ammonium chloride, Benzyl-cocoalkyl- dimethylammonium chlorides, Methyl benzethonium chloride, Benzethonium chloride, Cetylpyridinium chloride and Domiphen bromide.

11. Composition according to claim 7, characterized in that the amount of component d) is 0.001 to 15% by weight

12. A wet wipe liquor comprising a composition according to claim 1.

13. Use of a composition according to claim 1 for the preservation of technical or domestic products.

14. Use according to claim 13, characterized in that the technical product is selected from polymer dispersions, paints, adhesives, paper coating compositions, textile softening and sizing compositions, cleaning and polishing compositions, cosmetic and pharmaceutical preparations, household products, pack preservatives, spinning baths, cooling lubricants, leather treatment compositions, wet wipes and silicone and bitumen emulsions.

15. Technical or domestic product which comprises 0.01 to 1% by weight (of the composition according to claim 1.
